(19) United States
(12) Patent Application Publication
(10) Pub. No.: US 2020/0121294 A1
(43) Pub. Date: Apr. 23, 2020

(54) METHODS AND SYSTEMS FOR MOTION DETECTION AND COMPENSATION IN MEDICAL IMAGES

(71) Applicant: General Electric Company, Schenectady, NY (US)

(72) Inventors: Yelena Viktorovna Tsymbalenko, Mequon, WI (US); Thomas Andrew Kraus, Waukesha, WI (US)

(21) Appl. No.: 16/162,944

(22) Filed: Oct. 17, 2018

(51) Int. Cl. A61B 8/08; G06T 7/246; G06T 5/00; G06T 5/50; A61B 8/00; A61B 8/06

(52) U.S. Cl. CPC A61B 8/5276; G06T 7/248; G06T 5/003; G06T 5/50; A61B 8/488; G06T 2207/10024; A61B 8/469; A61B 8/06; A61B 8/5246; G06T 2207/10132; G06T 2207/20104; A61B 8/463

(57) ABSTRACT

Various methods and systems are provided for compensating for motion in medical images. As one example, a method for a medical imaging system may include independently tracking motion of a first object and motion of a second object across a plurality of image frames acquired with the medical imaging system, and for a selected image frame of the plurality of image frames, compensating the selected image frame for the motion of the first object and for the motion of the second object to generate a compensated selected image frame and outputting the compensated selected image frame for display on a display device, where the compensation for the motion of the first object is performed independently of the compensation for the motion of the second object.

### FIELD

Embodiments of the subject matter disclosed herein relate to medical imaging.

### BACKGROUND

Ultrasound, for medical or industrial applications, is an imaging modality that employs ultrasound waves to probe the acoustic properties of a target object (e.g., the body of a patient) and produce a corresponding image. When imaging a patient using ultrasound, motion due to patient breathing, patient heartbeat, or movement of the probe may cause image artifacts that may present as image blurring of organ and/or tissue.

### BRIEF DESCRIPTION

In one embodiment, a method for a medical imaging system includes independently tracking motion of a first object and motion of a second object across a plurality of image frames acquired with the medical imaging system, and for a selected image frame of the plurality of image frames, compensating the selected image frame for the motion of the first object and for the motion of the second object to generate a compensated selected image frame and outputting the compensated selected image frame for display on a display device, where the compensation for the motion of the first object is performed independently of the compensation for the motion of the second object.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasound imaging system according to an embodiment of the invention.

FIG. 2 is a flow chart illustrating a method for automatically detecting objects in medical images and tracking the motion of the detected objects.

FIG. 3 is a flow chart illustrating a method for performing motion compensation on medical images based on tracked motion of detected objects.

FIGS. 4-6 illustrate example ultrasound images including automatically identified objects.

### DETAILED DESCRIPTION

The following description relates to various embodiments of automatically identifying one or more objects present in a medical image and independently tracking motion of those objects across two or more consecutive images in order to apply targeted motion compensation techniques to the images, thereby reducing image artifacts. In some examples, a plurality of objects may be tracked at one time (e.g., two, three, four, or more objects in a single image frame may tracked across multiple successive image frames). The objects may include separate anatomical features, such as organs, lesions, blood vessels, etc., as well as sub-structures, such as heart chambers or multiple lesions within a single organ. The objects may be identified and tracked independently from one another, allowing identification of different levels of motion for different objects.

The targeted motion compensation techniques that are applied to the images may be selected based on the imaging mode used to obtain the images as well as the tracked motion of the detected objects. The motion compensation techniques may be applied in a targeted manner, such that different regions of the images including different identified objects having different motion may be subject to different motion compensation techniques or different motion compensation parameters. In doing so, image artifacts may be reduced in a manner that is most appropriate for the individual objects being tracked. The medical images to which the described object tracking and motion compensation may be applied may be obtained using ultrasound or another real-time or near real-time imaging modality. While examples of an ultrasound system and motion compensation of images obtained by the ultrasound system are presented below, the object tracking and motion compensation may be applied to other types of images, such as x-ray fluoroscopy images.

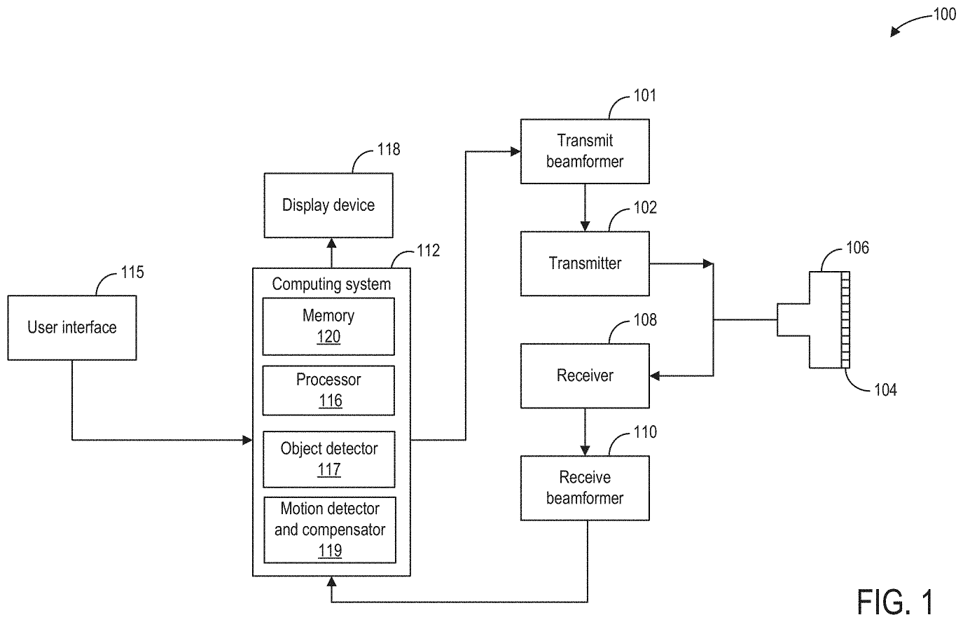

Turning now to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. The transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to one or more datasets acquired with an ultrasound imaging system. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a computing system 112 including a processor 116 and memory 120. Processor 116 controls the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain.

The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

Computing system 112 further includes resources (e.g., memory 120, processor 116) that may be allocated to store and execute an object detector module, referred to as object detector 117, and a motion detector and compensator module, herein referred to as motion detector and compensator 119. Object detector 117 is configured to analyze images to identify objects (e.g., anatomical features) present within the images. For example, object detector 117 may analyze each image frame acquired with the ultrasound imaging system 100 and identify anatomical features within each image frame, such as a heart, liver, lungs, blood vessels, and/or other organs, tissue, and/or structure. Each frame may be tagged with an indication of the object(s) identified in that image, where appropriate or desired. For example, an image including a view of a heart may be annotated to include an indication of the boundaries of the heart, such as in the form of box around the heart.

The object detector may be trained to detect a plurality of predefined objects (e.g., predefined anatomical features) using machine learning (e.g., deep learning), such as neural networking or other training mechanisms that are specific to object detection in a medical imaging environment. The object detector may be trained in a suitable manner. For example, object detector 117 may be trained to identify anatomical features typically associated with one or more different types of ultrasound exams, such as echocardiograms, fetal ultrasounds, and so forth. The training may include supplying a set of medical images of human anatomical features, in views typically obtained during the ultrasound exams, to object detector 117. Each image may be tagged (in a format readable by the object detector) with the anatomical features in the image. Further, at least in some examples, each anatomical feature may be annotated to indicate the boundaries/edges of that feature. Object detector 117 may then learn to identify anatomical features in patient medical images, as well as learn the boundaries of each anatomical feature. In some examples, the training of the object detector may include model-driven training concepts (e.g., where a mathematical 3D model of an anatomical feature of interest is used to train detection of the anatomical feature).

In some examples, the training of the object detector 117 may be stringent, such that the object detector 117 is trained to not only identify that one or more objects are present in a given image frame, but also trained to identify which anatomical features are represented by which objects. For example, the object detector 117 may be trained to determine that, in an example image frame, four objects are present in the frame. The object detector 117 may be further trained to identify which anatomical features correspond to which object, e.g., that the first object is a spleen, the second object is a kidney, the third object is a diaphragm, and that the fourth object is a lung. However, in other examples, the training of the object detector 117 may be less stringent, such that the object detector 117 may be able to determine the presence and boundaries of each separate object in a given image frame, but unable to identify which anatomical features correspond to which objects.

The training of the object detector 117 may allow the object detector 117 to track an identified object even as the object moves into and out of the imaging plane, thereby changing the size, shape, or other features of the identified object. For example, as the heart beats, a first image frame of the heart may include a view of an interior of one or more chambers of the heart while a second image frame may include a view of the cardiac muscle or other features instead of or in addition to the one or more chambers. The object detector 117 may be trained to determine that the anatomical features of the heart in the second image frame are still a part of the heart identified in the first image frame. However, in other examples, the object detector 117 may be trained to only identify that a tracked object is the same object across multiple image frames if the changes in object size, shape, or appearance resulting from movement of the object or the ultrasound probe are less than a threshold. For example, in the scenario presented above where the beating of the heart causes different anatomical features of the heart to be present in different image frames, the object detector 117 may determine that the different anatomical features of the heart present in the different image frames are different objects.

The object detector 117 may generate an indication of the position of each identified object in each image frame, and the motion detector and compensator 119 is configured to track the movement of objects detected by the object detector 117 across two or more frames of images and apply appropriate motion compensation to current and/or subsequent images based on the tracked movement. For example, the object detector 117 may identify a first object in a first image frame, such as an organ. The object detector 117 may generate a tracking boundary that defines the outer coordinates of the first object within an x,y coordinate system of the first image, at least for the purposes of tracking the first object. The tracking boundary may enclose the associated identified first object and may or may not intersect one or more portions of the identified first object (e.g., the tracking boundary may be rectangular and may intersect the top-most point of the first, bottom-most point of the first object, and each side-most point of the first object).

A tracking boundary may be associated with each identified object in an image frame. The tracking boundaries may have suitable geometries, such as square, rectangular, circular, polyhedron, etc. The geometries of the tracking boundaries may the same for each identified object, or the geometries of the tracking boundaries may be based on the geometries of the respective identified objects, such that different identified objects may be associated with tracking boundaries having different geometries. The tracking boundaries may be distinct from the boundaries of the identified objects, although in some examples, the tracking boundaries may at least partially track the boundaries of the associated identified objects.

For a subsequent, second image frame, the object detector 117 may similarly identify the first object in the second image frame, and if the first object has moved, the position of the tracking boundary is updated to track the movement of the first object. The motion detector and compensator 119 may compare the position of the tracking boundary in the second image frame to the position of the tracking boundary in the first image frame and determine a motion score based on a difference between the position of the tracking boundary in the second image frame and the position of the tracking boundary in the first image frame. By tracking the change in position of the tracking boundary rather than the change in position of the identified object, the motion tracking may be simplified and require fewer processing resources (e.g., compared to systems that determine motion in an image frame based on pixel brightness changes from frame to frame). Further, the use of the tracking boundaries provides for individual, independent object motion tracking.

The motion score represents the change in position of the tracking boundary and hence the identified object (e.g., organ) within a fixed, two-dimensional coordinate system (e.g., defined by the edges of the image frame). The motion score may take a suitable form. In some examples, the motion score may be a relative score that represents a level of movement of the identified object, such as a low level of movement, a medium level of movement, and a high level of movement. For example, the change in position of the tracking boundary may be represented by a movement value that includes the sum of an absolute value of a change in an x coordinate of the tracking boundary and the absolute value of a change in a y coordinate of the tracking boundary (where the x,y coordinate of the tracking boundary is at a corner of the tracking boundary, a center of the tracking boundary, or other suitable point of the tracking boundary, so long as the same point of the tracking boundary is tracked across multiple image frames). In other examples, the movement value may be the higher of the absolute value of the change in the x coordinate of the tracking boundary and the absolute value of the change in the y coordinate of the tracking boundary. A movement value that is greater than zero but below a first threshold may be classified as a low level of movement, a movement value that is between the first threshold and a second, higher threshold may be classified as a medium level of movement, and a movement value that is above the second threshold may be classified as a high level of movement. In other examples, the motion score may include the actual change in position of the tracking boundary (e.g., the movement value described above). Further, in examples where a change in volume occurs to the object and/or the object moves into or out of the imaged plane, the identified object in the second image frame may be larger or smaller than the identified object in the first image frame. In such examples, the size of the tracking boundary for the identified object in the second image frame may be adjusted relative to the size of the tracking boundary in the first image frame. The movement of the identified object may be based on the change in coordinates of the tracking boundary and/or based on a change in size/scale of the tracking boundary.

A separate motion score may be calculated for each identified object in the second image frame. By separately calculating motion scores for each identified object, objects that have different levels of movement (e.g., organs close to the heart versus organs further away from the heart) may be assigned motion scores that accurately reflect that object's level of movement.

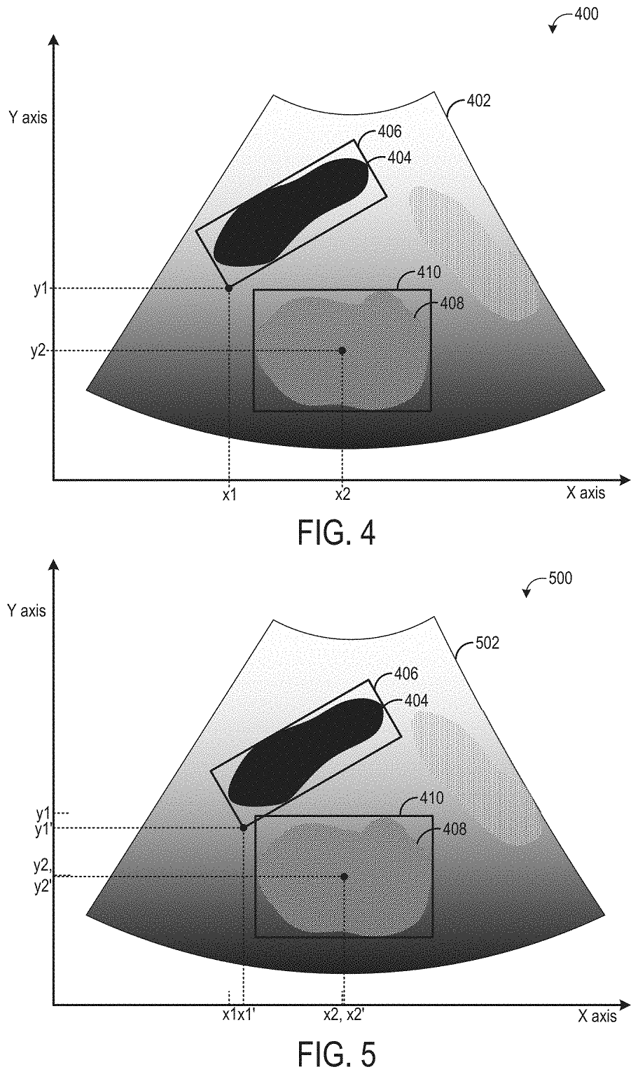

FIG. 4 shows a schematic illustration 400 of a first image frame 402 including identified objects with tracking boundaries having positions defined by an x,y coordinate system. For example, first image frame 402 may be obtained by the ultrasound imaging system 100. The object detector 117 may identify one or more objects present in the first image. As shown, two objects have been detected by the object detector, a first object 404 and a second object 408. Each detected object may be associated with a respective tracking boundary. The first object 404 is associated with a first tracking boundary 406 while the second object 408 is associated with a second tracking boundary 410. As appreciated from FIG. 4, each tracking boundary is sized and positioned based on the identified object with which the tracking boundary is associated. For example, each side of the first tracking boundary 406 intersects with an edge of the first object 404 and no portion of the first object 404 lies outside of the tracking boundary 406. Each tracking boundary may be oriented based on an orientation of the associated identified object, e.g., the first object 404 may have a longitudinal axis and the first tracking boundary 406 may have a longitudinal axis that is parallel with the longitudinal axis of the first object 404.

The position of each identified object may be defined based on the coordinates of the respective associated tracking boundary, relative to a fixed coordinate system, such as the x,y coordinate system shown in FIG. 4 (which may be defined by the first image frame 402). The coordinates of the tracking boundaries may be determined in a suitable manner. For example, the position of the first tracking boundary 406 may be defined by the coordinates of a corner point of the tracking boundary, e.g., the lower left point. Herein, the lower left point of the first tracking boundary 406 may be positioned at x1,y1 on the coordinate system. In another example, the position of the second tracking boundary 410 may be defined by the coordinates of a center point of the second tracking boundary. Herein, the center point of the second tracking boundary 410 may be positioned at x2,y2 on the coordinate system. Further, each tracking boundary may be defined by the size and orientation of the tracking boundary. For example, the first tracking boundary 406 may have a height of 16 mm, a width of 44 mm, and an angle of 30° relative to the x axis. The second tracking boundary 410 may have a height of 29 mm, a width of 43 mm, and an angle of 0° relative to the x axis. As appreciated from FIG. 4, both tracking boundaries have substantially similar geometries, as each tracking boundary is a rectangle.

As the identified objects move due to patient motion (e.g., breathing, heart beat) and/or movement of the ultrasound probe, the identified objects may be tracked across subsequent image frames to calculate motion scores for each identified object. FIG. 5 shows a schematic illustration 500 of a second image frame 502 defined by the same coordinate system as shown in FIG. 4. The second image frame 502 may be obtained by the ultrasound system 100 subsequent to the first image frame 402; for example, the first and second image frames may be consecutively obtained image frames. As such, the second image frame 502 is imaging the same imaging subject as the first image frame 402.

The second image frame 502 includes the same objects identified in the first image frame 402, including the first object 404 and the second object 408. The second image frame 502 likewise includes the respective tracking boundaries, including the first tracking boundary 406 and the second tracking boundary 410. From the first image frame 402 to the second image frame 502, both the first object 404 and the second object 408 have moved, and accordingly, each of the tracking boundaries is moved along with the identified objects. Thus, the coordinates of the first tracking boundary 404 are now x1′,y1′ and the coordinates of the second tracking boundary are now x2′,y2′. By comparing the coordinates of the first tracking boundary 406 in the second image frame 502 to the coordinates of the first tracking boundary 406 in the first image frame 402, a motion score for the first object 404 may be calculated. For example, if the x and y axes are each in mm, the absolute value of the difference between x1′ and x1 may be 3.5 mm, the absolute value of the difference between y1′ and y1 may be 3.5 mm, and a movement value may be 7 mm, which is the sum of the absolute values. The motion score may be set as the movement value, or the motion score may be a relative level of motion based on the movement value compared to one or more thresholds. For example, a movement value of 0.1-2.9 mm may be a low motion score, a movement value of 3-4.9 mm may be a medium motion score, and a movement value of 5 mm or greater may be a high motion score. Thus, the first object 404 may be classified as having a high motion score. In contrast, while the second object 408 has also moved, the movement value of the second object 408 may be relatively low, such as 0.45 mm, and thus the second object 408 may be classified as having a low motion score.

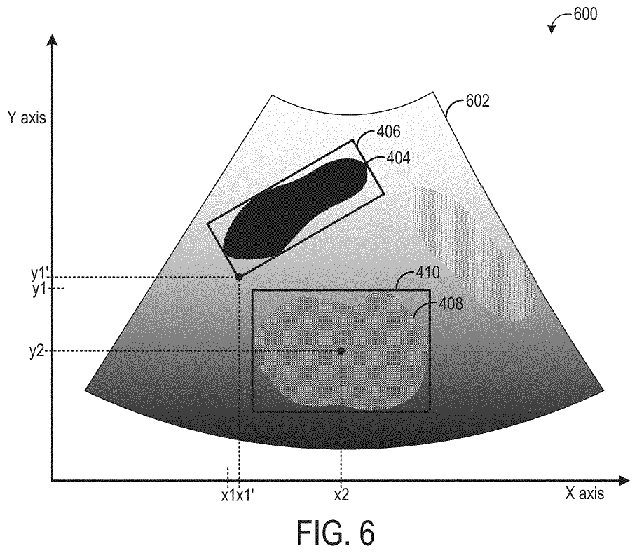

FIG. 6 shows a schematic illustration 600 of a third image frame 602 defined by the same coordinate system as shown in FIG. 4. The third image frame 602 may be obtained by the ultrasound system 100 subsequent to the first image frame 402; for example, the first and third image frames may be consecutively obtained image frames. As such, the third image frame 602 is imaging the same imaging subject as the first image frame 402.

The third image frame 602 includes the same objects identified in the first image frame 402, including the first object 404 and the second object 408. The third image frame 602 likewise includes the respective tracking boundaries, including the first tracking boundary 406 and the second tracking boundary 410. From the first image frame 402 to the third image frame 602, the first object 404 has moved, and accordingly, the first tracking boundary 406 is moved along with the first object 404. Specifically, the first object 404 has moved out of the imaging plane (e.g., the x-y plane, where the first object 404 has moved along the z axis perpendicular to the x and y axes). Thus, while the first object 404 has not moved along the x or y axes, less of the first object 404 is present in the imaging plane, and therefore the first object 404 appears smaller in size in the third image frame relative to the first image frame. Accordingly, the scale of the first tracking boundary 406 in the third image frame 602 has changed relative to the scale of the first tracking boundary in the first image frame 402. For example, the first tracking boundary in the third image frame 602 may have a height of 15 mm and a width of 40.5 mm, which are each smaller than the respective dimensions of the first tracking boundary 406 in the first image frame 402. The tracking coordinates of the first tracking boundary have also changed due to the change in size of the tracking boundary, from x1,y1 to x1′,y1′. In the third image frame 602, the second object 408 has not moved relative to the first image frame 402. The motion score for the first object 404 may be determined as described above, e.g., based on the absolute value of the change in both the x and y coordinates, or the motion score may be calculated based on the change in size of the first tracking boundary.

Returning to FIG. 1, the motion detector and compensator 119 may apply one or more motion compensation processes to the acquired image information for each image frame where object movement is detected. The motion compensation applied to a given image frame may depend on the motion score for each detected object as well as the imaging mode used to acquire the imaging information, and in some examples, based on the type of object that is identified (e.g., the anatomical feature represented by the identified object). For example, differential frame averaging may be performed for standard B-mode imaging, where the weighting of the averaging may be different for regions of high motion than regions of low or no motion. In Doppler flowing imaging, the Doppler range gate may be automatically sized, placed, and/or steered relative to a region of interest (ROI) based on the motion scores of objects in and/or out of the ROI. In another example, motion compensation may be applied to some types of anatomical features to reduce blurring, where motion of the object provides no clinical value (e.g., a gallbladder), while no motion compensation may be applied to other types of anatomical features where motion of the object may provide clinical value (e.g., a beating heart). After applying the motion compensation, a motion compensated image may be output for display and/or storage.

For example, referring to the example image frames illustrated in FIGS. 4 and 5, when the second image frame 502 is processed for display, one or more motion compensation techniques may be applied to the second image frame in order to reduce blurring, flash, or other artifacts arising from motion during imaging. The motion compensation techniques may be applied in a targeted manner based on the identified objects and corresponding motion scores. For example, the image that is output for display may include the brightness values for each pixel of the second image frame 502 averaged with the brightness values for each pixel of the first image frame 402. However, the averaging may be adjusted based on the motion scores calculated for each identified object. As one example, all the pixels within the first tracking boundary 406 of the second image frame 502 may be averaged with the pixels of a corresponding area of the first image frame 402, where the averaging is performed with a first weighting, while all the pixels within the second tracking boundary 410 of the second image frame 502 may be averaged with the pixels of a corresponding area of the first image frame 402, where the averaging is performed with a second weighting. In some examples, all pixels outside any tracking boundaries may be averaged with a third weighting. For example, for a pixel a in the resulting image, a brightness value of the pixel a may be calculated based on the brightness of the pixel a2 in the second image frame and the brightness of the pixel a1 in the first image frame. Because the pixel a2 is in the first tracking boundary 406 of the second image frame and thus has a high motion score, the brightness value of the pixel a in the resulting image may be calculated using a first weight according to the following equation:

a=a2(1)+a1(0) Eq. 1

Because of the high motion, only the brightness values for the pixels in the second image frame may be represented, which may reduce blurring. For a pixel b in the resulting image, a brightness value of the pixel b may be calculated based on the brightness of the pixel b2 in the second image frame and the brightness of the pixel b1 in the first image frame. Because the pixel b is in the second tracking boundary 410 of the second image frame and thus has a low motion score, the brightness value of the pixel b in the resulting image may be calculated using a second weight according to the following equation:

b=b2(0.75)+b1(0.25) Eq. 2

For a pixel c in the resulting image, a brightness value of the pixel c may be calculated based on the brightness of the pixel c2 in the second image frame and the brightness of the pixel c1 in the first image frame. Because the pixel c is outside any detected object and thus has no motion score, the brightness value of the pixel c in the resulting image may be calculated using a second weight according to the following equation:

c=c2(0.5)+c1(0.5) Eq. 3

It is to be understood that the above-described equations are merely exemplary, and other methods for performing frame averaging and adjusting the frame averaging based on motion scores are possible. Further, though an ultrasound system is described by way of example, it should be understood that the present object detection, motion detection, and motion compensation techniques may also be useful when applied to images acquired using other imaging modalities, such as x-ray fluoroscopy. The present discussion of an ultrasound imaging modality is provided merely as an example of one suitable imaging modality. Further, separate object detector and motion detector and compensator modules were described above, but it should be appreciated that the object detection, motion detection, and motion compensation techniques described herein may be performed by a single module or multiple modules, and that the modules may be stored and/or executed on a single device (e.g., computing system 112) or stored and/or executed across multiple devices and/or the cloud. Further still, while the object detection and motion scores described above were described with respect to two-dimensional images, the object detection and motion scores may be performed on three-dimensional volume data.

As used herein, the terms “module” or “device” may include a hardware and/or software system that operates to perform one or more functions. For example, a module or device may include a computer processor, controller, or other logic-based device that performs operations based on instructions stored on a tangible and non-transitory computer readable storage medium, such as a computer memory. Alternatively, a module or device may include a hard-wired device that performs operations based on hard-wired logic of the device. Various modules or units shown in the attached figures may represent the hardware that operates based on software or hardwired instructions, the software that directs hardware to perform the operations, or a combination thereof.

“Modules” or “devices” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be appropriately programmed or instructed to perform operations described herein from the instructions described above. Additionally or alternatively, one or more of these devices may be hard-wired with logic circuits to perform these operations.

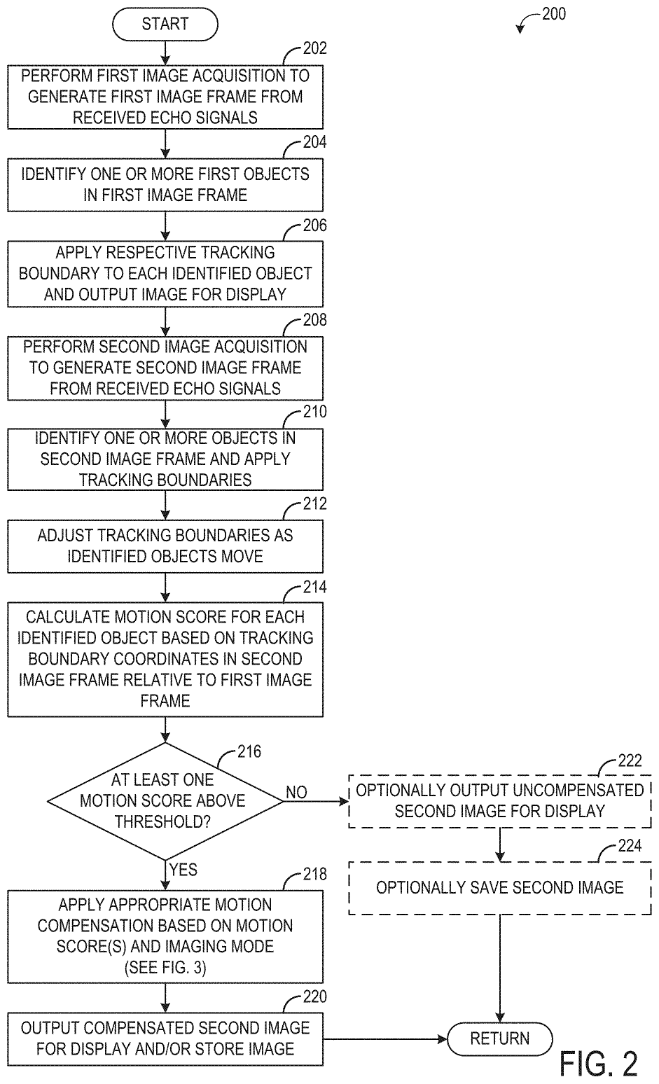

Turning now to FIG. 2, a method 200 for object tracking and motion compensation is presented. Method 200 and the other methods described herein may be executed by a computing system (such as computing system 112 shown in FIG. 1) according to instructions stored on a non-transitory memory of the system (e.g., memory 120 shown in FIG. 1) in combination with the various signals received at the computing system (e.g., echo signals received from receiver 108). The computing system may employ a display device (such as display device 118 shown in FIG. 1) to display ultrasound images, according to the methods described below. The methods included herein will be described with regard to an ultrasound probe, although it should be understood that image information acquired from other imaging modalities could be used without departing from the scope of the methods.

At 202, a first image acquisition is performed to generate a first image frame from received echo signals. For example, the transducer elements of the ultrasound probe may be activated (e.g., voltage may be applied) to emit ultrasonic signals into a body (e.g., of a patient). The ultrasonic signals are back-scattered from structures within an interior of the body to produce echoes that return to the transducer elements, and the echoes are converted into electrical signals, or ultrasound data, by the transducer elements that are received by a receiver and/or a receive beamformer that output ultrasound data. The ultrasound data may include image data which includes image values, such as intensity/brightness values for B-mode ultrasound or power values (or a power component) for Doppler mode ultrasound. A 2D image may then be generated from the acquired ultrasound imaging data.

At 204, one or more objects in the first image frame are detected. As explained above with respect to FIG. 1, an object detector executing on the computing system (e.g., object detector 117) may detect each object present in the first image frame, where the objects may be anatomical features such as organs, tissue, and/or other structures. The object detector may be trained to only detect a predefined set of objects and not detect other features that may be present in the first image (such as non-structural features like fluid or gas), or the object detector may be trained to detect the presence of any definable feature.

At 206, a respective tracking boundary for each detected object is applied to the first image frame and the first image frame is output for display and/or is stored in memory. The applied tracking boundaries may be visible in the image that is output for display and/or stored. By including visible tracking boundaries in the displayed image, an operator of the ultrasound probe or other clinician may view the detected objects, confirm that the size and/or placement of the tracking boundaries are correct, and/or adjust the position of the ultrasound probe to better visualize a desired anatomical feature. Further, when the object detector is trained to actually identify the anatomical feature associated with each object (e.g., identify that a detected object is a liver), an annotation identifying the anatomical feature of the object may also be included in the image. In this way, the operator or other clinician may be able to learn the relative position and appearance of various anatomical features. In other examples, the tracking boundaries may be transparent or the coordinates of the tracking boundaries may be determined and stored in memory but not actually applied to the image itself. Each tracking boundary may define the size and position of the underlying/associated detected object, and as such may be sized to fit the associated detected object.

At 208, a second image acquisition is performed to generate a second image frame from received echo signals. As explained above with respect to FIG. 1, the ultrasound system may acquire data at a suitable frame-rate, such as 10 Hz to 30 Hz (e.g., 10 to 30 frames per second). The second image acquisition may be performed similarly to the first image acquisition described above. At 210, one or more objects in the second image frame are identified and associated tracking boundaries are applied. The objects in the second image frame may be identified similarly to the one or more objects identified in the first image frame, e.g., with the object detector. One or more of the objects detected in the second image frame may the same as one or more of the objects as detected in the first image frame, e.g., if a liver was identified in the first image frame, the liver may similarly be identified in the second image frame. Further, the object detector may determine if the second image frame includes one or more objects not present in the first image frame. Additionally, in some examples, the object detector may be configured to use the first image frame as a reference and may detect changes to the one or more objects from the first image frame to the second image frame, e.g., if a first object has changed in size and/or position, the object detector may be configured to determine the change to the first object. Any objects detected in the second image frame that were not present in the first image frame may be associated with a tracking boundary, and any tracking boundaries associated with objects in the first image frame that are no longer present in the second image frame may be removed.

At 212, one or more tracking boundaries may be adjusted as one or more of the identified objects moves in the second frame relative to the first frame. For example, if a first object detected in the first image frame has changed position in the second image frame due to patient motion or movement of the ultrasound probe, the tracking boundary associated with that first object may be adjusted to track the movement of the first object. Any tracking boundaries associated with objects that are stationary in the second image frame may likewise remain stationary.

At 214, a motion score is calculated for each identified object in the second image frame based on the tracking boundary coordinates in the second image frame relative to the first image frame. For example, if a first object detected in the first image frame is also present in the second image frame, the tracking boundary coordinates of that first object in the second image frame may be compared to the coordinates of that tracking boundary in the first image frame. Each identified object in the second image frame may be assigned a motion score. If an identified object in the second image frame is in the same position as in the first image frame (e.g., the coordinates for the tracking boundary for that identified object are the same in both image frames), the identified object may be assigned a motion score of zero, or a relative motion score of “no motion,” where no motion may include no detectable movement, or small amounts of movement within a tolerable range (e.g., 0.1 mm of movement or less). However, if the coordinates for a tracking boundary of an identified object have changed from the first image frame to the second image frame, the identified object may be assigned a non-zero motion score. The motion score may be an actual value (e.g., movement in mm) or the motion score may be a relative score (e.g., low, medium, or high), as explained above with respect to FIG. 1. The motion score may be calculated similarly to the motion score determination described above with respect to FIG. 1, e.g., by a motion detector and compensator executing on the computing system.

At 216, method 200 includes determining if at least one motion score is above a threshold. If the motion scores are numeric, a motion score may be above the threshold when the motion score is greater than zero or greater than a motion threshold that allows for small movements to go undetected and/or that allows for measurement errors, such as 0.1 mm. If the motion scores are relative levels of motion, a motion score other than “no motion” may be above the threshold (e.g., all of low, medium, and high motion scores may be above the threshold). The motion score threshold may be fixed, or the motion score threshold may change depending on the type of detected object. For example, a heart may have a different motion score threshold than a liver.

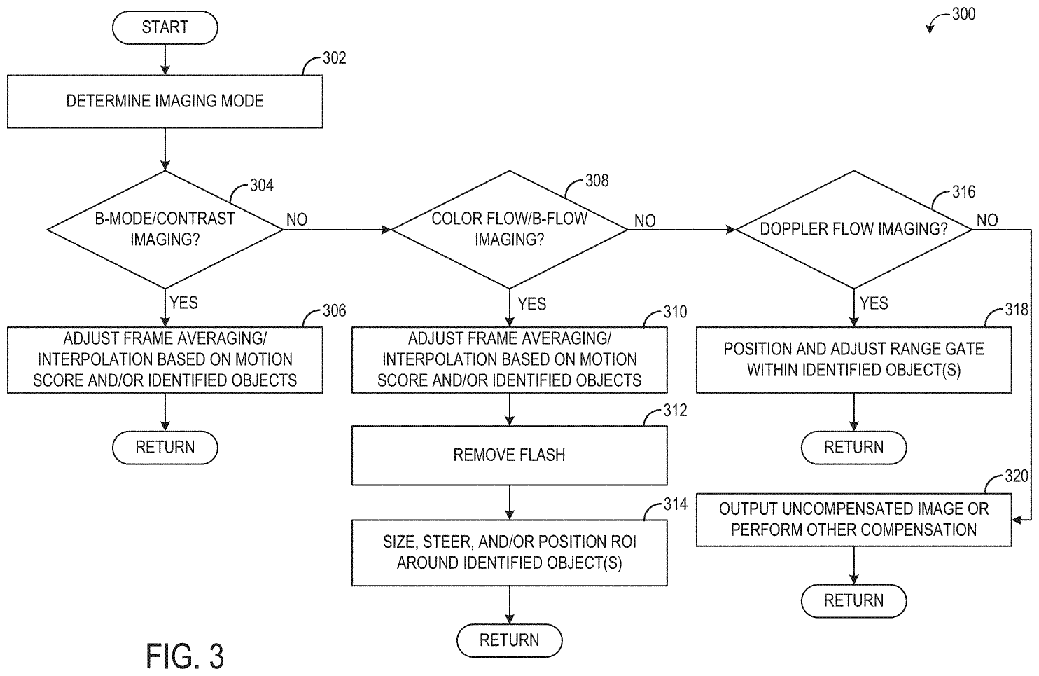

If at least one object has an associated motion score that is above the threshold, method 200 proceeds to 218 to apply one or more appropriate motion compensation techniques to the second image frame based on the motion score(s) and the current imaging mode. The motion compensation(s) that may be performed will be explained in more detail below with respect to FIG. 3. Briefly, the motion compensation(s) may be performed to reduce or eliminate image artifacts associated with patient and/or probe motion during imaging. Different motion compensation techniques may be applied for different imaging modes (e.g., frame averaging may be adjusted for B-mode imaging while range gate placement and/or size may be adjusted for Doppler imaging), and thus the selection of which motion compensation techniques to apply to the second image frame may depend on the current imaging mode. Further, the type or level of the motion compensation techniques performed may also depend on the motion score(s). For example, when the imaging mode dictates that frame averaging be applied to compensate for the detected motion, the weighting of the frame averaging may be based on the motion score(s). When more than one object is detected in the second image frame, the frame averaging may be targeted, such that the frame averaging performed in a region around a first object with a first motion score may be performed with a first weighting, while the frame averaging performed in a region around a second object with a second motion score may be performed with a second, different weighting.

At 220, a second, compensated image is output for display and/or for storage. The second image may be compensated by the one or more motion compensation techniques that are selected based on the imaging mode and motion score(s). For example, the second image may be compensated by differential frame averaging or interpolation. By detecting motion in each identified object independently, and then compensating for the detected motion independently, appropriate motion compensation techniques may be applied to the image, thereby reducing blurring, flash, and/or other artifacts.

While method 200 is described herein as determining motion scores based on two image frames, and then performing motion compensation on the second image frame, it is to be understood that any suitable number of image frames may be included in the motion score calculation and/or the motion compensation. For example, a motion score for a detected object may be determined based on a change in position and/or size of the detected object across three, four, or more image frames. Further, the motion score may be calculated as a rate of change rather than an absolute movement value, and thus may take into account how fast the object is moving in addition to how much the object is moving. Further still, the frame averaging and/or interpolation described herein (and in more detail below with respect to FIG. 3) to compensate for detected motion may include averaging more than two frames, such as averaging three, four, or more frames.

Returning to 216, if it is determined that no motion scores are above the threshold, method 200 proceeds to 222 to optionally output an uncompensated second image for display. As used herein, an uncompensated image may include an image that has not been compensated for motion, as no motion has been detected by the motion detector and compensator described herein. However, an uncompensated image may have other image processing techniques applied. The display of the second image may be optional because the second image may be substantially similar to the first image, and thus the computing system may save storage and/or processing resources by not displaying the second image. Likewise, method 200 may optionally save the second image at 224. The saving of the second image may be optional, as the computing system may instead delete the second image, given that the second image may not provide clinical value. Method 200 then returns.

FIG. 3 is a flow chart illustrating a method 300 for performing motion compensation based on imaging mode and one or more motion scores of one or more identified objects. Method 300 may be performed as part of method 200, for example in response to a determination that one or more identified objects in an image frame has a motion score above a threshold. Accordingly, method 300 may be performed by a computing system, such as computing system 112 of FIG. 1, configured to execute a motion detector and comparator, as described above with respect to FIGS. 1 and 2.

At 302, the current imaging mode is determined. The current imaging mode may refer to the imaging mode via which the image information was acquired during the second image acquisition. Additionally or alternatively, the current imaging mode may refer to the processing of the image information that was acquired during the second image acquisition in order to generate the second image frame. As explained above with respect to FIG. 1, the ultrasound system may be configured to operate in one or more imaging modes and/or process acquired image information in one or more modes depending on the ultrasound exam being conducted and/or the diagnostic information being obtained during the exam. Different imaging modes may provide different information regarding the subject being imaged. For example, B-mode imaging may provide standard 2D grayscale images typically used in diagnostic exams such as fetal ultrasounds, echocardiograms, lesion detection, and so forth. Doppler imaging may be used to visualize and/or measure moving fluid, such as blood flow. The determination of which imaging mode(s) is currently being employed may be based on user input (e.g., an operator of the ultrasound system may enter user input via a user interface (such as user interface 115 of FIG. 1) indicating which imaging mode(s) are to be utilized), which type of ultrasound probe is coupled to the ultrasound system/computing system (e.g., transducer array configuration), how the ultrasound probe is being controlled by the corresponding computing system (e.g., the pulse sequence, frequency, etc., of the signals output by the transducer elements of the ultrasound probe), and so forth.

At 304, method 300 determines if the ultrasound system is currently operating in a B-mode or contrast imaging mode. During B-mode (also referred to as brightness mode) ultrasound, the transducers simultaneously scan a plane through the imaging subject that can be viewed as a two-dimensional image. During contrast imaging, a contrast agent is injected into the bloodstream of the patient being imaged and B-mode images may be obtained. Contrast mode imaging may utilize a reduced acoustic power setting relative to B-mode imaging, with different scan sequencing using a phase inversion, for example, and algorithms to reduce/eliminate certain visual features that may normally be seen under B-Mode imaging, thus highlighting the minute vascularity within the tissue being imaged. The contrast agent contains micro-bubbles that are carried through the vascular and capillary system which increases the reflected signal back to the transducer. In this way, the objects potentially being detected, such as a lesion, are further brought out or highlighted over traditional B-Mode imaging.

If the current imaging mode is B-mode or contrast imaging, method 300 proceeds to 306 to adjust the frame averaging and/or interpolation based on the motion score(s) determined at 214 of method 200 and/or based on the identified objects. The frame averaging may include averaging each pixel value of the first image frame and the corresponding pixel value of the second image frame, on a pixel-by-pixel basis. For example, the brightness value of the first pixel of the first image frame may be averaged with the brightness value of the first pixel of the second image frame, and the brightness value of the first pixel of the second image frame may be replaced with the averaged brightness value. The averaging may be weighted, such that the brightness value for the second image frame contributes more or less to the second image than the brightness value of the first image frame. Adjusting the frame averaging may include adjusting the weighting, e.g., increasing or decreasing the weighting based on the motion score and/or type of identified object. For example, if a motion score is low, the first and second image frames may be given equal weight in the frame averaging, while if the motion score is high, the second image frame may be given more weight. In another example, if the identified object is a heart, the second image frame may be given more weight than if the identified object is a liver.

Further, the adjustment to the averaging may be performed independently and in a targeted manner based on the different identified objects and different motion scores. For example, a first identified object may have a first motion score, and a second identified object may have a second motion score. The averaging of the pixels in a first region of the second image frame that includes the first object may be performed with a first weighting while the averaging in a second region of the second image frame that includes the second object may be performed with a second weighting that is different than the first weighting. A similar technique may be applied to frame interpolation, where interpolation of pixels in a first region of the second image frame that includes the first object may be performed differently than the interpolation of pixels in a second region of the second image frame that includes the second object. Method 300 then ends.

Returning to 304, if the current imaging mode is not B-mode or contrast imaging, method 300 proceeds to 308 to determine if the current imaging mode is color flow or B-flow imaging. Color flow imaging is a type of Doppler ultrasound used to measure and/or visualize blood flow that produces a color-coded map of Doppler shifts superimposed onto a B-mode ultrasound image. During color flow imaging, the transducer elements are controlled in a pulsed fashion. B-flow imaging is a non-Doppler imaging mode that provides real-time imaging of blood flow by digitally encoding the outgoing ultrasound beam, then decoding and filtering the returning beam.

If the current imaging mode is color flow or B-flow imaging, method 300 proceeds to 310 to adjust the frame averaging and/or interpolation based on the motion score(s) and/or identified objects, similar to the frame averaging/interpolation adjustments described above for the B-mode/contrast imaging modes. Further, in some examples, flash may be removed, as indicated at 312. Flash artifacts are the presence of a color signal in color flow imaging of color B-flow imaging that may be caused by tissue motion rather than the movement of interest (e.g., blood flow). If an identified object has a certain motion score (e.g., a motion score above a flash threshold, such as a medium or high motion score), and that object is not the target of the color flow imaging (e.g., the identified object is an organ, soft tissue, a cyst, or otherwise not within a user defined region of interest), flash may be removed by maintaining the underlying grayscale, B-mode imaging derived pixels in the region of the identified object with the high motion score and overriding any color pixels that would otherwise be displayed in the region of the identified object. In other examples, flash artifacts may be removed by simply not displaying the image that would otherwise include the flash artifact. For example, if a motion score of an identified object in the current image frame is high enough, the current image frame may be discarded and the previous image frame may be maintained on the display device.

Additionally or alternatively, when imaging in color flow or B-flow imaging modes, the region of interest (ROI) around an identified object of interest (e.g., a blood vessel) may be automatically sized, positioned, and/or steered based on the motion score and tracking boundary for the identified object of interest, as indicated at 314. For example, during color flow imaging, the user (e.g., sonographer) may designate a ROI within which the color flow imaging may be performed. The object detector may identify an object that overlaps with the ROI and generate a tracking boundary for the identified object defining the size, shape, and/or position of the identified object. The motion detector and comparator may determine the motion score of the identified object. The motion detector and comparator may then adjust the size, shape, and/or position of the ROI so that the ROI falls within the identified object and so that the ROI tracks the movement of the identified object. Further, if the motion score of the identified object is higher (e.g., high motion), the size of the ROI may be decreased relative to if the motion score were lower (e.g., low motion), which may assist in maintaining the ROI within the boundary of the identified object even as the identified object moves. Method 300 then returns.

Returning to 308, if the current imaging mode is not color flow or B-flow, method 300 proceeds to 316 to determine if the current imaging mode is a Doppler flow imaging mode. Doppler flow imaging may include any type of imaging that relies on Doppler, other than color flow imaging, such as Power Doppler imaging. Power Doppler, also referred to as Doppler or PW imaging, is typically represented on the display as a signal magnitude on a timeline scale whereas Color Doppler, also referred to as color flow herein, is displayed overlaid on the tissue greyscale image (e.g., B-mode image) and is updated in real time as a frame rate. Color flow provides an average velocity for the selected region of interest (ROI) whereas Power Doppler provide a more precise velocity value over a sample gate within the image represented on a time-based display.

If the current imaging mode is Doppler flow imaging, method 300 proceeds to 318 to position and adjust the range gate within a targeted identified object based on the tracking boundary and/or motion score. For example, the user may indicate a target region of interest/object that overlaps with an object identified by the object detector. The motion detector and compensator may automatically place and/or size the range gate appropriately for the identified object of interest (e.g., vessel). Further, the size of the range gate may be adjusted based on the motion score, e.g., if the motion score is low, the size of range gate may be increased to produce a more complete signal, while if the motion score is high, the size of the range gate may be smaller and the range gate may be steered to keep the range gate inside the target object. Method 300 then returns.

If the current imaging mode is not Doppler flow, some other imaging mode may be currently in use, such as strain elastography (SE) or shear wave elastography (SWE). Accordingly, method 300 proceeds to 320 to output an uncompensated image, or to apply some other compensation that may or may not be motion-based. In SWE, B-mode imaging and Doppler may be utilized for an automated push pulse and color flow for tracking, so as to highlight the elasticity of the objects being tracked. In SE, instead of using Doppler, the user mechanically provides a push. In either SE or SWE, motion compensation may be applied in a similar manner as described above, such as by placing and steering a region of interest. Further, in some examples, one or more of the above-described imaging modes may be used during an interventional procedure, such as a biopsy or for directed radiation therapy, where knowing the exact position of an object of interest is important. In the example of directed radiation therapy, the motion of a detected object (e.g., a lesion that is receiving the radiation therapy) may be tracked using the object detection and motion tracking techniques described herein, and the radiation therapy system may be adjusted based on the tracked motion (e.g., the radiation beam may be turned off when the detected object is outside a window where the radiation beam intersects a patient, or the radiation beam may be moved to follow the detected object). Method 300 then returns.

Thus, methods 200 and 300 illustrated in FIGS. 2 and 3 and described above provide for automatic object detection, motion detection, and compensation of the detected motion in order to reduce motion related image artifacts. When imaging patients using ultrasound, motion due to patient breathing, patient heart rate, movement of the probe, etc., may cause image artifacts that show up as image blurring, for example. To be able to properly evaluate and diagnose the patient scans, being able to minimize image artifacts such as blurring and flash artifacts and increasing the accuracy of visualization and interventional procedures is desirable.

Thus, the methods described herein utilize artificial intelligence (AI) for real-time object detection (OD) within acquired ultrasound images to identify organs/tissue/structure, to then track subsequent AI OD organ/tissue/structure to determine motion parameters, and then utilize the motion parameters to apply targeted motion compensation for ultrasound. The AI OD motion parameters may then be applied to pre-defined organ/tissue/structure limitation ranges to be able to apply specific targeted motion compensation per tracked organ/tissue. Such predefined organ/tissue/structure limitation ranges may be categorized into low/mid/high (for example) compensation techniques. In this way, each targeted AI OD motion compensation and artifact removal can be applied independently since each organ/tissue/structure is tracked independently.

Knowing the targeted organ/tissue/structure location and motion parameters (also referred to herein as motion scores), various targeted motion compensation techniques may be applied. When imaging in B-mode and/or contrast imaging mode, the motion compensation may include applying targeted frame averaging and/or frame interpolation based upon the motion score that is obtained by the AI OD per organ/tissue/structure. When imaging in color flow (CF) or B-flow imaging modes, the motion compensation may include applying targeted frame averaging and/or frame interpolation based upon the motion score that is obtained by the AI OD per organ/tissue/structure. Further, the motion compensation may include removal of undesirable artifacts, such as flash, which are associated with the motion. Additionally, knowing the organ/tissue/structure along with the motion score(s), the motion compensation may include the automatic placement and adjustment of the ROI around the organ/tissue/structure of interest, such as sizing, steering, and placing the ROI automatically and appropriately, based upon the AI OD organ/tissue/structure tracking boundary and adjusting the ROI based upon the motion score. By doing so, the frame rate of the imaging may be optimized to optimize the CF visualization. When imaging in Doppler flow imaging mode, the motion compensation may include the automatic placement and adjustment of the Doppler range gate within the targeted AI OD organ/tissue/structure of interest. Adjustment of the range gate could be inclusive of the sample volume size as well as the steering angle to optimize the Doppler image.

In addition to the image-mode specific motion compensation described above, the automatic object detection and motion score calculation may be used to improve other aspects of the ultrasound imaging. For example, the object detection and motion score calculation may allow for selective and optimal storage by limiting or reducing the amount of image storage necessary if the motion is limited or non-existent. The frame data that is not changing does not provide any additional clinical information and value, therefore it could be eliminated from the storage. In another example, the registration of the ultrasound image frame and another image (e.g., an image obtained via magnetic resonance imaging) may be improved by knowing the motion score within the volume being acquired. During an interventional procedure targeting a particular organ/tissue/structure, such as for ablation, it is important to know the precise position of the organ/tissue/structure within relation to the biopsy needle. Having the ability to track and compensate motion, or provide the motion score, allows for greater precision and accuracy of the procedure. To improve biopsy needle visualization and tracking, the targeted AI OD organ/tissue/structure motion score could be used to more accurately project the needle path as well as minimize the visualization artifacts due to motion. Additionally, the object detection and motion score calculation described herein may provide the ability to display an alternate heart rate graph based upon motion-based organ/tissue/structure detection. Additionally, while scanning, a motion quality score or indicator could be displayed to assist the sonographer/radiologist to minimize motion imaging artifacts.

As described above, the tracking boundaries associated with identified objects may be displayed for educational purposes. Having already identified the organ/tissue/structure utilizing the AI OD, the organ/tissue/structure may be visualized by displaying bounding outline(s) (ROIs) along with visual identification of such organ/tissue/structure in real-time. Being able to quickly identify would allow a beginner or inexperienced user to visualize organ/tissue/structure that is actively being scanned in real time. Additionally, having the AI OD tracking boundary visualization, it would provide the ability to the sonographer/radiologist to adjust the transducer position in relationship to the targeted scan organ/tissue/structure for optimal imaging.

A technical effect of automatically detecting objects, tracking objection motion, and motion compensating images based on the tracked objects and object motion is reduced motion-based image artifacts such as image blurring.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for a medical imaging system, comprising:
independently tracking motion of a first object and motion of a second object across a plurality of image frames acquired with the medical imaging system; and
for a selected image frame of the plurality of image frames,
compensating the selected image frame for the motion of the first object and for the motion of the second object to generate a compensated selected image frame; and
outputting the compensated selected image frame for display on a display device, where the compensation for the motion of the first object is performed independently of the compensation for the motion of the second object.

2. The method of claim 1, wherein independently tracking motion of the first object and motion of the second object across the plurality of image frames comprises:
associating a first tracking boundary with the first object;
associating a second tracking boundary with the second object;
tracking the motion of the first object by tracking motion of the first tracking boundary across the plurality of image frames; and
tracking the motion of the second object by tracking motion of the second tracking boundary across the plurality of image frames.

3. The method of claim 1, wherein compensating the selected image frame for the motion of the first object and for the motion of the second object comprises:
applying a first motion compensation parameter to at least a first region of the selected image frame, the first motion compensation parameter selected based on a relative level of the motion of the first object; and
applying a second motion compensation parameter to at least a second region of the selected image frame, the second motion compensation parameter selected based on a relative level of the motion of the second object.

4. The method of claim 3, wherein the medical imaging system comprises an ultrasound imaging system configured to operate in a plurality of imaging modes, wherein the first motion compensation parameter and second motion compensation parameter are each further selected based on a current imaging mode of the ultrasound imaging system.

5. A method for a medical imaging system, comprising:
automatically detecting a first object and a second object in an image frame acquired with the medical imaging system;
assigning a first motion score to the first object based on a size and/or position of the first object in the image frame relative to a size and/or position of the first object in a prior image frame acquired with the medical imaging system;
assigning a second motion score to the second object based on a size and/or position of the second object in the image frame relative to a size and/or position of the second object in the prior image frame;
processing the image frame, the processing including applying a first motion compensation parameter to the image frame based on the first motion score and applying a second motion compensation parameter to the image frame based on the second motion score; and
outputting the processed image frame for display on a display device.

6. The method of claim 5, further comprising associating the first object with a first tracking boundary defining a size and position of the first object in the image frame and associating the second object with a second tracking boundary defining a size and position of the second object in the image frame.

7. The method of claim 6, wherein assigning the first motion score comprises determining a change in size and/or position of the first tracking boundary from the prior image frame to the image frame and assigning the first motion score based on the change in size and/or position of the first tracking boundary, and wherein assigning the second motion score comprises determining a change in size and/or position of the second tracking boundary from the prior image frame to the image frame and assigning the second motion score based on the change in size and/or position of the second tracking boundary.

8. The method of claim 7, wherein determining a change in size of the first tracking boundary comprises determining first dimensions of the first tracking boundary in the prior image frame and determining second dimensions of the first tracking boundary in the image frame, and wherein assigning the first motion score comprises assigning the first motion score based on a difference between the first dimensions and the second dimensions, where the first dimensions and second dimensions are each determined relative to a fixed coordinate system.

9. The method of claim 7, wherein determining a change in position of the first tracking boundary comprises determining first coordinates of the first tracking boundary in the prior image frame and determining second coordinates of the first tracking boundary in the image frame, and wherein assigning the first motion score comprises assigning the first motion score based on a difference between the first coordinates and the second coordinates, where the first coordinates and second coordinates are each determined relative to a fixed coordinate system.

10. The method of claim 5, wherein:
applying the first motion compensation parameter to the image frame based on the first motion score comprises averaging brightness values of pixels in the image frame with brightness values of pixels in the prior image frame according to a first weight in a region of the image frame that includes the first object, the first weight selected based on the first motion score; and
applying the second motion compensation parameter to the image frame based on the second motion score comprises averaging brightness values of pixels in the image frame with brightness values of pixels in the prior image frame according to a second weight in a region of the image frame that includes the second object, the second weight selected based on the second motion score, the first weight different than the second weight.

11. The method of claim 5, wherein the medical imaging system is an ultrasound system operating in a color flow mode or a B-flow mode, and wherein applying the first motion compensation parameter to the image frame based on the first motion score comprises:
receiving a user input identifying a region of interest (ROI) that overlaps the first object in the prior image frame;
adjusting a size and/or position of the ROI in the image frame based on the first motion score; and
performing color flow imaging or B-flow imaging in the ROI.

12. The method of claim 11, wherein applying the second motion compensation parameter to the image frame based on the second motion score comprises averaging brightness values of pixels in the image frame with brightness values of pixels in the prior image frame in a region of the image frame that includes the second object, a weight of the averaging selected based on the second motion score.

13. The method of claim 5, wherein the medical imaging system is an ultrasound system operating in a Doppler flow mode, and wherein applying the first motion compensation parameter to the image frame based on the first motion score comprises:
receiving a user input identifying a Doppler flow imaging target that overlaps the first object in the prior image frame;
positioning a range gate for the prior image frame based on the position of the first object in the prior image frame;
adjusting the range gate for the image frame based on the first motion score; and
performing Doppler flow imaging according to the adjusted range gate.

14. The method of claim 13, wherein adjusting the range gate for the image frame based on the first motion score comprises decreasing a size of the range gate as a relative level of motion indicated by the first motion score increases.

15. The method of claim 5, wherein the first object is a first anatomical feature and the second object is a second anatomical feature that is different than the first anatomical feature, and wherein the first motion compensation parameter is different than the second motion compensation parameter.

16. The method of claim 15, wherein the first motion score is different than the second motion score.

17. An ultrasound system, comprising:
an ultrasound probe including an array of transducer elements;
a display device; and
a computing system with computer readable instructions stored on non-transitory memory that when executed during operation of the ultrasound system, cause the computing system to:
automatically detect a first object and a second object in a first image frame generated from data acquired with the ultrasound probe;
automatically detect the first object and the second object in a second, subsequent image frame generated from data acquired with the ultrasound probe;
assign a first motion score to the first object based on a position of the first object in the first image frame relative to a position of the first object in the second image frame;
assign a second motion score to the second object based on a position of the second object in the first image frame relative to a position of the second object in the second image frame;
process the second image frame, including applying a first motion compensation parameter to the second image frame based on the first motion score and applying a second motion compensation parameter to the second image frame based on the second motion score; and
output the processed second image frame for display on the display device.

18. The system of claim 17, wherein the computer readable instructions, when executed, cause the computing system to:
associate a first tracking boundary with the first object in the first image frame and the second image frame;
associate a second tracking boundary with the second object in the first image frame and in the second image frame;
assign the first motion score based on a change in position of the first tracking boundary from the first image frame to the second image frame; and
assign the second motion score based on a change in position of the second tracking boundary from the first image frame to the second image frame.

19. The system of claim 17, wherein the first motion score is different than the second motion score, and wherein the first motion compensation parameter is different than the second motion compensation parameter.

20. The system of claim 19, wherein the processing of the second image frame includes averaging pixel brightness values of the second image frame with pixel brightness values of the first image frame, wherein the first motion compensation factor comprises a first weight being applied to the averaging in a first region of the second image frame, and wherein the second motion compensation factor comprises a second weight being applied to the averaging in a second region of the second image frame, the first region including the first object and the second region including the second object, and where the first weight is different than the second weight.
